(19) United States
(12) Patent Application Publication
(10) Pub. No.: US 2005/0143760 A1
(43) Pub. Date: Jun. 30, 2005

(54) Endoscopic gastric constriction device

(76) Inventor: Mir A. Imran, Los Altos Hills, CA (US)

Correspondence Address: Susan Schmitt;PETERS, VERNY, JONES & SCHMITT LLP, Suite 230, 425 Sherman Avenue, Palo Alto, CA, 94306, US

(21) Appl. No.: 10/991,648

(22) Filed: Nov. 18, 2004

Related U.S. Application Data
(63) Continuation-in-part of application No. 10/295,115, filed on Nov. 14, 2002.
Application No. 10/295,115 is a division of application No. 09/847,884, filed on May 1, 2001, now Pat. No. 6,535,764.

(51) Int. Cl. A61B 17/10

(52) U.S. Cl. 606/142

(57) ABSTRACT

An endoscopically implantable device is provided that cinches the stomach together to create a reduced diameter for a portion of the stomach. The device may be, for example, a gastrorestrictive device used to treat overweight or obese patients. The device may be used, for example, to treat gastroesophogeal reflux disease (GERD).

### RELATED APPLICATION DATA

This application is a continuation in part of U.S. application Ser. No. 10/295,115, filed Nov. 14, 2002, which is a divisional of U.S. application Ser. No. 09/847,884 filed May 1, 2001 all of which are incorporated herein by reference.

### FIELD OF THE INVENTION

The invention relates to a gastric constriction device for restricting the diameter of a portion of a stomach or creating a small pouch in the stomach.

### BACKGROUND

A variety of devices and techniques have been used to surgically alter the digestive tract of a patient in order to treat obesity and related diseased. The techniques include creating a small pouch in a patient's stomach that restricts emptying(restrictive procedures), bypassing a portion of the digestive tract, e.g. or the stomach or small intestines (malabsorptive procedures), or a combination of both.

Most of these techniques involve substantial surgery where portions of the stomach are stapled closed and/or where the gastrointestinal tract is shortened by rerouting portions of the gastrointestinal tract to reduce absorption of nutrients. These surgical procedures are expensive and create substantial risks to the patient either during surgery, in recovery or with respect to subsequent failure of the procedure.

The Lap Band® procedure, a less invasive procedure, has been used where a band is laparoscopically delivered to the outside of the stomach. In the Lap Band® procedure, the band is positioned around an upper portion of the stomach and tightened to create a small stomach pouch. The band may be inflatable so that the diameter of passageway exiting the small pouch is adjustable.

However, the Lap Band® procedure still requires tunneling through the abdomen to the stomach in a laparoscopic procedure. Also, the laparoscopic banding devices and procedures have had complications that result in stomach perforation.

Accordingly, it would be desirable to provide an improved gastric restrictive device and procedure.

In treating gastroesophogeal reflux disease, a procedure known as a fundal plication is performed which brings the stomach wall together just below the lower esophageal sphincter (LES). This is typically performed in an open or laparoscopic procedure.

Accordingly, it would be desirable to provide a less invasive or alternative procedure for treating GERD.

### SUMMARY OF THE INVENTION

The present invention provides a device that cinches the stomach together to create a reduced diameter for a portion of the stomach. The present invention also provides such a device that may be implanted endoscopically, i.e., through the esophagus into the stomach. The present invention also provides a gastrorestrictive device that may be used to treat overweight or obese patients. The present invention also provides a device that may be used to treat gastroesophogeal reflux disease (GERD) by using a device to endoscopically perform a procedure with a similar result as a fundal plication, i.e., a reduced stomach diameter near the LES.

According to one aspect of the invention anchors are endoscopically deployed in the stomach. Cinching mechanisms such as, e.g., a wire or tether, are attached to the anchors. The cinching mechanisms and are used to draw portions of the stomach together to create a reduced diameter for at least a portion of the stomach. The cinching mechanisms may be tied together with a connector. The taughtness of the cinching mechanisms or the size of the reduced stomach diameter may be adjustable.

### DESCRIPTION OF THE DRAWINGS

FIG. 1 illustrates a device implanted in the stomach in accordance with the invention.

FIG. 2 illustrates the device implanted in the stomach of FIG. 1 with the stomach cinched to form a reduced diameter.

FIG. 3 is an enlarged view of a portion of the device of FIG. 1.

FIG. 4 is a top view of a device in accordance with the invention.

FIG. 5A is a top view of a device in accordance with the invention.

FIG. 5B is an enlarged view of the connecting ring of the device of FIG. 5A.

FIG. 5C is an enlarged view of a portion of the connecting ring of FIG. 5C with a connecting wire.

FIG. 6 illustrates the device of FIGS. 5A-5C being implanted in the stomach.

FIG. 7A is a front view a device in accordance with the invention, implanted to treat gastroesophogeal reflux disease.

FIG. 7B is a side view of the device of FIG. 7A.

### DETAILED DESCRIPTION

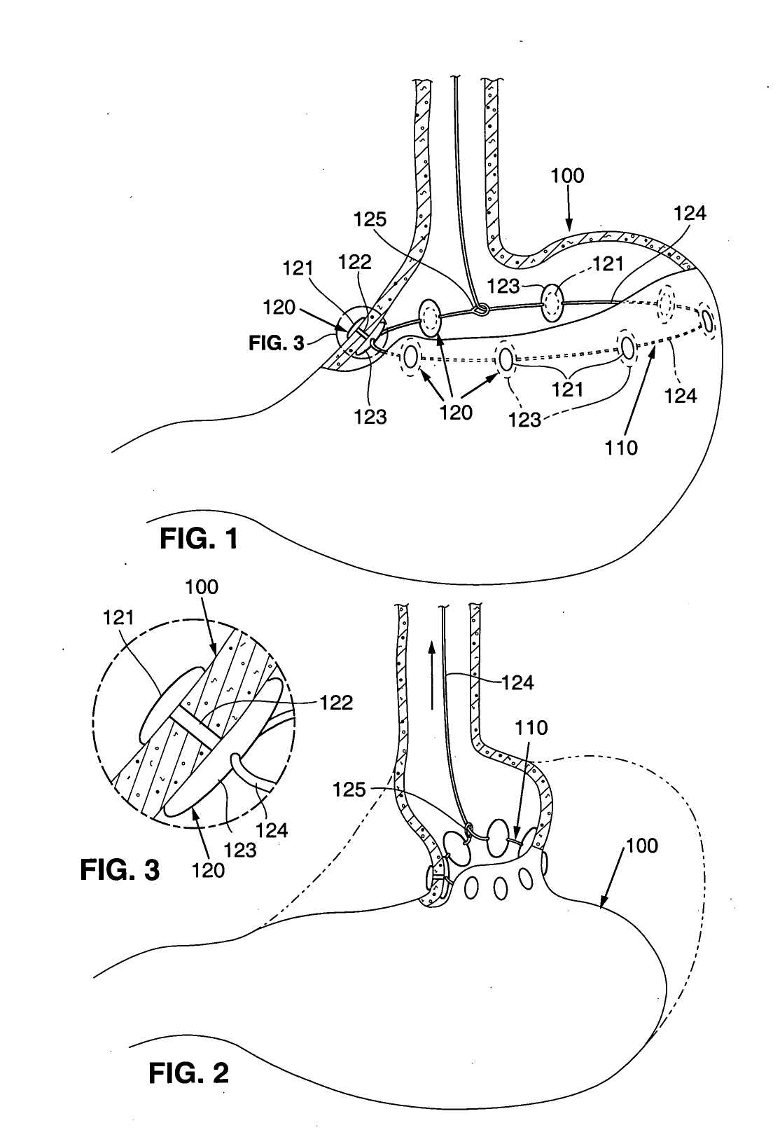

Referring to FIGS. 1-3 a device 110 in accordance with the invention is illustrated. The device 110 comprises a plurality of anchors 120 attached to the wall of a stomach 100.

As illustrated in FIG. 3, each anchor 120 comprises an end portion 121 configured to interface with the outside of the stomach wall hold the anchor 120 in place. The end portion 121 has a sufficient surface area with respect to forces applied to the anchor to prevent device pull out. An elongate portion 122 extends from the end portion 121 into the stomach 100. A connecting device 123 is coupled inside the stomach to the elongate portion 122.

As illustrated in FIG. 1, a tether 124 extends through each connecting devices 123 (e.g., through a hole). The tether 124 is then joined in a loop with one end of the tether extending through a loop 125 in the other end of the tether and extending out of the stomach.

As illustrated in FIG. 2, the tether 124 used to draw the stomach wall together. This creates a small pouch for receiving food and a small diameter conduit for passing food from the pouch. Thus a feeling of satiety of fullness is sensed after ingestion of a smaller amount of food. The tether 124 is secured in the cinched position e.g. with a securing device in the loop 125 or by crimping the tether 124 ends together at the loop 125 and trimming the excess length of the tether extending out of the stomach.

The anchors 120 may be implanted endoscopically through the esophagus. For example, the end portion 121 may be delivered by way of a cannula or hollow needle from the inside of the stomach and through the stomach wall. Examples of attaching device to the inside of the stomach wall are described in related co-pending U.S. application Ser. No. 10/295,115, filed Nov. 14, 2002, which is a divisional of U.S. application Ser. No. 09/847,884 filed May 1, 2001, incorporated herein by reference.

FIG. 4 illustrates a variation of the device 200 in accordance with the invention. Anchors 220a-f comprise end portions 221a-f interfacing with the outside of the stomach wall, and tethers 224a-f extending from the end portions 221a-f respectively through the stomach wall 101 and into the stomach 100. Anchors 220a, 220b, anchors 220c, 220d, and anchors 220e, 220f are respectively positioned across from each other to form pairs that draw the stomach together in a folded configuration. Accordingly, tethers 224a, 224b are connected with connector 225; tethers 224c, 224d are connected with connector 226; and tethers 224e, 224f are connected with connector 227. The tethers 224a-f may be drawn together as illustrated and the excess length trimmed.

The anchors 220a-f may be implanted endoscopically in a manner similar to anchors 120 described herein.

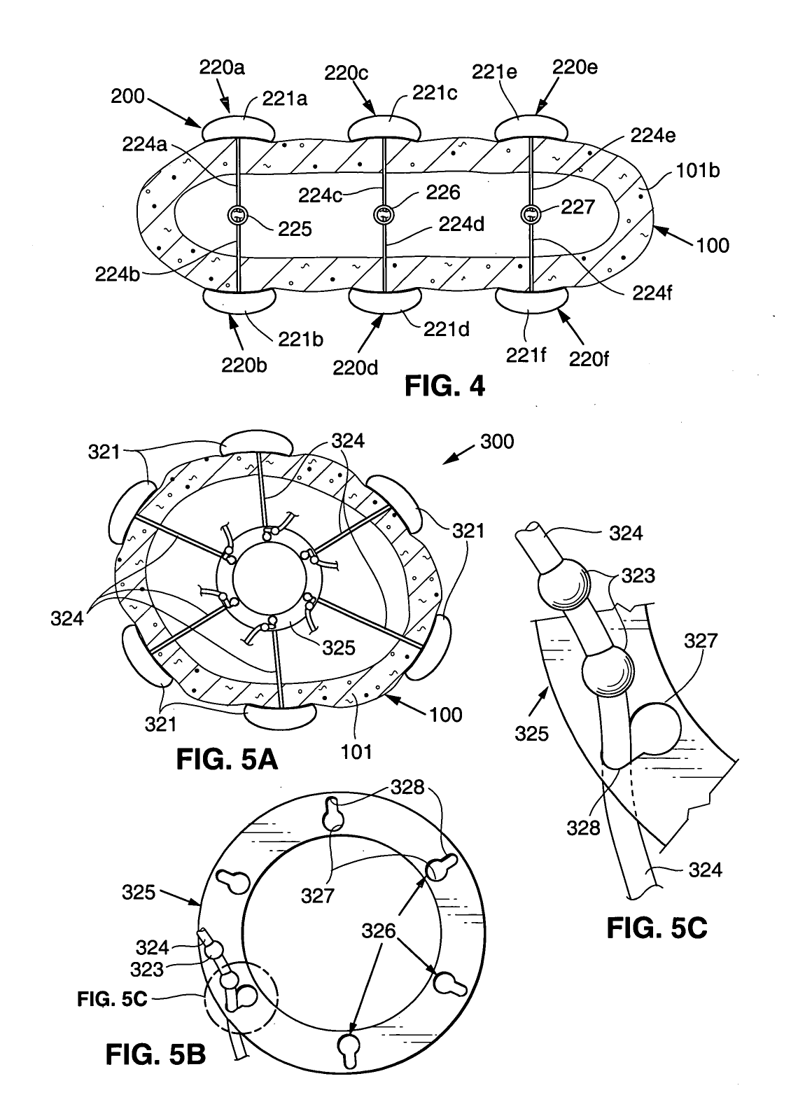

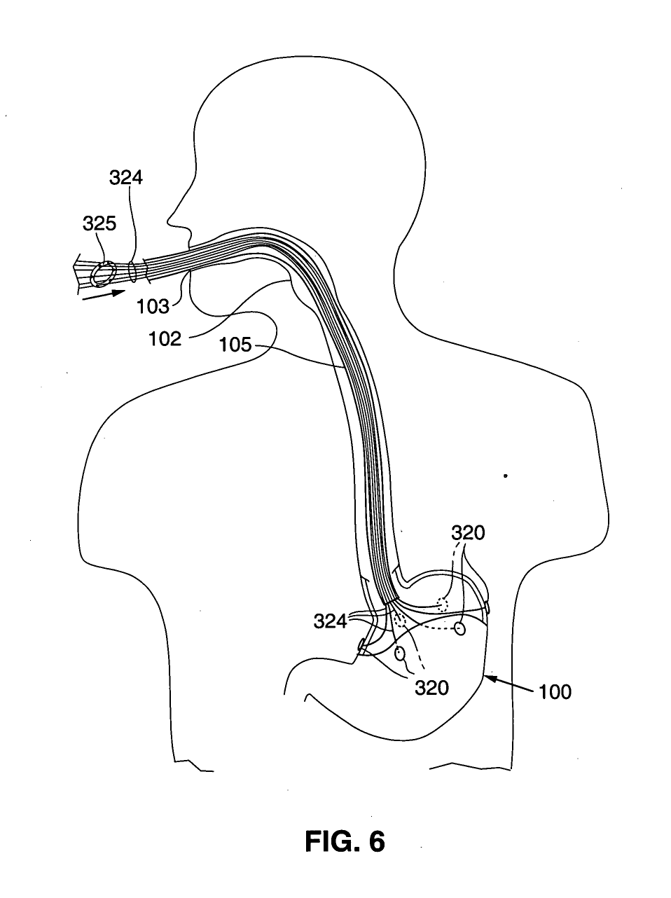

FIGS. 5A-6 illustrate another variation of a device 300 in accordance with the invention. Anchors 320 are arranged circumferentially about the stomach 100. Anchors 320 include end portions 321 interfacing with the outside of the stomach wall, and tethers 324 extending from the end portions 321 through the stomach wall 101 and into the stomach 100. Tethers 324 are connected to the connecting ring as shown in FIG. 5C.

Connecting ring 325 comprises a plurality of openings 326 extending around the circumference of the ring 325. The tether 324 includes a plurality of ball members 323 staggered along the length of the tether 324. Each of the tethers 324 are positioned through the openings 326 in the ring 325. The openings 326 each comprise a wider diameter portion 327 through with the ball members 323 are sized to pass, and a narrow portion 328 that are narrower than the ball members 323. The tethers 324 are pulled through the openings 326 in the ring 325 to cinch the stomach wall. When the desired diameter opening is achieved, the tethers 324 are secured or locked into the ring 325 by positioning the tether 324 through the narrow portion 328 of the opening. The tether 324 may be further secured in position with other mechanical safety features such as, e.g., hooks or stops that move or rotate into a position that prevents the tether from moving from the narrow postion 328 to the wider diameter portion 327.

FIG. 6 illustrates the implanting of the device 300 described with respect to FIGS. 5A-5C. The anchors 320 are implanted endoscopically in a manner similar as anchors 120 described herein. The tethers 324 extend from the stomach 100 and out through the esophagus 102. Each of the tethers 324 may be numbered or otherwise identified with reference to their position about the stomach 100. The tethers are inserted through the openings 326 in the ring outside of the patient's mouth 103. The ring is sized so that it may fit through endoscope 105 that extends into the stomach 100. The ring 325 is then pushed through the endoscope 105 into the stomach while holding the ends of the tethers 324 outside of the mouth. Each of the tethers 324 are pulled, drawing the stomach inward until the desired diameter opening is formed in the stomach 100. Each tether 324 is then secured to the ring 325 and the tethers are trimmed to remove at least a portion of the extra length. The tethers 324 and ring 325 are configured so that the taughtness of the cinched stomach may be adjusted. Leaving a portion of a length of a tether allows the device to be loosened and the diameter of the opening through the stomach formed by the device can be increased. Similarly the device may be tightened so that the diameter of the opening through the stomach is decreased. This may be done using endoscopically placed instruments.

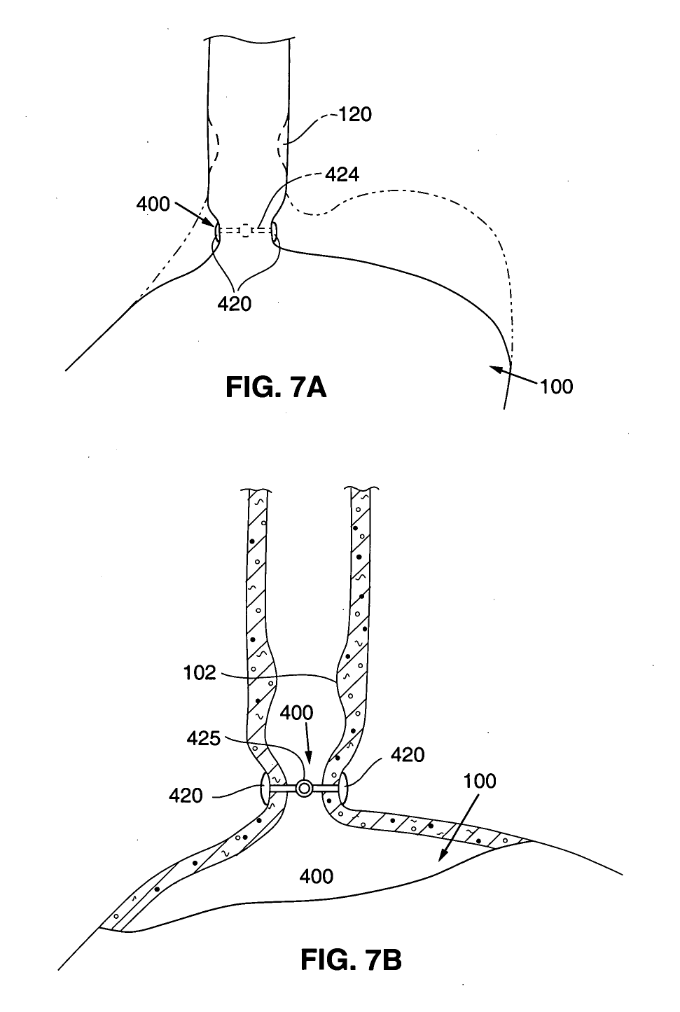

FIGS. 7A and 7B illustrate a device 400 in accordance with the invention that is being used to treat gastroesophogeal reflux disease (GERD). One or more pairs of opposing anchors 420 are implanted at a location just below the lower esophageal sphincter 102 and in the stomach 100. Anchors 420 include tethers 424 that are connected with connector 425 in a manner similar to that described herein with reference to anchors 220a-f and tethers 224a-f illustrated in FIG. 4. This provides relief to the lower esophageal sphincter in a manner similar to that of a fundal plication procedure typically used to treat GERD.

While this invention has been described with reference to preferred embodiments thereof, it will be apparent to one skilled in the art that various modifications and changes can be made without departing from the scope of the invention.

1. A device for reducing a diameter of at least a portion of a stomach comprising:
a plurality of anchors configured to be coupled to a stomach from within the stomach;
a cinching mechanism coupled to the plurality of anchors and configured to cinch the stomach to provide a reduced diameter of at least a portion of the stomach.

2. The device of claim 1 wherein the cinching mechanism comprises at least one tether coupled to at least one of the plurality of anchors.

3. The device of claim 1 wherein the anchors each comprise an end portion configured to be positioned outside of a stomach wall, a narrow portion coupled to the elongate portion and configured to extend through the stomach wall into the stomach.

4. The device of claim 3 wherein the cinching mechanism is coupled to the narrow portion.

5. The device of claim 4 wherein the cinching mechanism comprises at least one tether.

6. The device of claim 5 wherein the cinching mechanism further comprises a connector configured to be coupled to the at least one tether to draw the walls of the stomach together.

7. The device of claim 4 wherein the cinching mechanism comprises a connector coupled to the narrow portion.

8. The device of claim 7 wherein the cinching mechanism comprises a tether coupled to the connector and configured to draw the wall of the stomach together.

9. The device of claim 1 wherein the device is configured to treat gastroesophageal reflux disease.

10. The device of claim 1 wherein the device is configured a gastrorestrictive device.

11. The device of claim 1 wherein the diameter of the at least a portion of the stomach is adjustable with the cinching mechanism.

12. The device of claim 1 wherein the diameter of the at least a portion of the stomach is readjustable with the cinching mechanism.

13. A method for reducing the diameters of at least a portion of a stomach comprising:
providing a cinching mechanism for reducing the diameter of the at least a portion of the stomach;
endoscopically attaching the cinching mechanism to the inside of a stomach; and
drawing walls of the stomach inwards with the cinching mechanism to provide a reduced diameter for at least a portion of the stomach.

14. The method of claim 13 wherein the cinching mechanism is used to provide a gastrorestrictive device to treat obesity.

15. The method of claim 13 wherein the step of endoscopically attaching the device comprises endoscopically attaching the device adjacent the LES to treat GERD.

16. A device for reducing the diameter of a stomach comprising:
an anchor means for coupling the device to a stomach from within the stomach; and
a cinching means for drawing the walls of at least a portion of the stomach together to reduce the diameter of the at least a portion of the stomach.

17. The device of claim 16 wherein the device comprises means for treating obesity.

18. The device of claim 16 wherein the device comprises means for treating GERD.

19. A method for treating GERD comprising the steps of:
reducing the diameter of a stomach below the LES from within the stomach.
